(19) United States
(12) Patent Application Publication
(10) Pub. No.: US 2019/0029391 A1
(43) Pub. Date: Jan. 31, 2019

(54) COSMETIC KIT SYSTEM

(71) Applicant: Hannah Bennett, Vancouver, WA (US)

(72) Inventor: Hannah Bennett, Vancouver, WA (US)

(21) Appl. No.: 16/105,908

(22) Filed: Aug. 20, 2018

Related U.S. Application Data
(60) Provisional application No. 62/522,188, filed on Jun. 20, 2017.

(51) Int. Cl. A45D 33/00; A45D 40/28; G09B 19/00; A45D 34/04; A61Q 17/04; A61Q 19/04; A61K 8/35; A61K 8/60

(52) U.S. Cl. CPC A45D 33/00; A45D 40/28; G09B 19/0076; A45D 34/042; A61Q 17/04; A45D 40/20; A61K 8/35; A61K 8/60; A45D 2200/25; A45D 2200/1018; A61Q 19/04

(57) ABSTRACT

A cosmetic system including a first-applicator and a second-applicator. The first-applicator includes a first-composition including at least one sunscreen-agent. The second-applicator includes a second-composition including at least one skin-darkening agent. The cosmetic system provides users with a three-step system for highlighting and contouring different areas of the face and body.

### CROSS REFERENCE TO RELATED APPLICATION

The present application is related to and claims priority to U.S. Provisional Patent Application No. 62/522,188 filed Jun. 20, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cosmetics and more specifically relates to a cosmetic kit for naturally contouring and highlighting a user.

### 2. Description of Related Art

Contouring different areas of the face and body with makeup has become extremely popular with consumers. People will spend hours each week and hundreds of dollars on beauty products to achieve a contoured look. Having to constantly visit a tanning salon, beauty parlor, or other business to achieve these looks can become incredibly time-consuming and expensive. People with busy lifestyles may be unable to obtain a desired look and feel disappointed with how he/she looks while on summer vacation, bodybuilding, and the like. An effective solution is desired.

U.S. Pat. No. 6,409,997 to Mauricio Castro relates to a wax cosmetic stick. The described wax cosmetic stick includes a cosmetic stick composition comprising (a) a skin care agent and (b) a solid wax, wherein the composition comprises less than 1%, by weight, of oil, and methods of using such composition.

### BRIEF SUMMARY OF THE INVENTION

In view of the foregoing disadvantages inherent in the known cosmetics art, the present disclosure provides a novel cosmetic kit system. The general purpose of the present disclosure, which will be described subsequently in greater detail, is to provide users with a three-step system for highlighting and contouring different areas of the face and body. the system is for strategic tanning in which one uses tanning bronzers/tan intensifiers/tanning developer and SPF to be used together or on their own with the intentions of getting lasting highlight and/or contour on the face and/or body.

A cosmetic system is disclosed herein. The cosmetic system includes a first-applicator which may have a first-composition therein and a first applicator-end. The first applicator-end may be configured to apply the first-composition to at least one first-area on at least one body-part of a user. The first-composition may include at least one sunscreen-agent. Similarly, a second-applicator may have a second-composition therein and a second applicator-end. The second applicator-end may be configured to apply the second-composition to at least one second-area on the at least one body-part of the user and the second-composition may include at least one skin-darkening agent.

A method of using cosmetic system is also disclosed herein. The method of using cosmetic system may comprise the steps of: providing the cosmetic system; applying the first-composition to the at least one first-area on the at least one body-part of the user; and applying the second-composition to the at least one second-area on the at least one body-part of the user.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a cosmetic kit system, constructed and operative according to the teachings of the present disclosure.

FIG. 1 is a front perspective view of the cosmetic system during an ‘in-use’ condition, according to an embodiment of the disclosure.

FIG. 2A is a front perspective view of the cosmetic system of FIG. 1, according to an embodiment of the present disclosure.

FIG. 2B is a front perspective view of the cosmetic system of FIG. 1, according to an embodiment of the present disclosure.

FIG. 3A is a front perspective view of the cosmetic system of FIG. 1, according to an embodiment of the present disclosure.

FIG. 3B is a front perspective view of the cosmetic system of FIG. 1, according to an embodiment of the present disclosure.

FIG. 4 is a front perspective view of the cosmetic system of FIG. 1, according to an embodiment of the present disclosure.

FIG. 5A is a front perspective view of the cosmetic system of FIG. 1, according to an embodiment of the present disclosure.

FIG. 5B is a front perspective view of the cosmetic system of FIG. 1, according to an embodiment of the present disclosure.

FIG. 6 is a flow diagram illustrating a method of use for cosmetic system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cosmetics and more particularly to a cosmetic kit system as used to improve the cosmetic kit for naturally contouring and highlighting a user.

Generally, disclosed is a system which may utilize SPF and bronzers/tan intensifiers/tanning developer on the higher and lower regions of the face and body to simulate the look of higher cheek bones, sharp jaw bones, tighter abs, and much more. This may allow users to obtain a natural highlight and contour look without the need for makeup. As designed, it functions as a preparation tool for summer activities, bodybuilding competitions, vacations, and more. The present invention eliminates the need to visit expensive tanning salons and skincare centers in order to obtain a contoured and highlighted look to different areas of the body.

The system may include a set of products used to add natural and lasting highlights and contours to different areas of the face and body. The products may be provided in a tube, roller, stick, other applicator. A first applicator may include a low concentration of SPF to be used on higher points of the face, protecting sensitive skin around the eyes and to build a natural highlight. A second applicator may include a higher concentration of SPF to highlight the center of each previously treated area, providing a more concentrated highlight. A third applicator may be a tan-enhancing component that can be added to areas a user would like contoured. All three products may allow for highlighting and contouring without the need for makeup or expensive salon visits.

The third applicator may include self-tanning chemicals such as but not limited to DHA and/or erythrulose to be used on the portions of the face and/or body on which one wants to achieve a lasting contoured effect. The first and second applicator may include physical/mineral, chemical or broad-spectrum sun screen protection to be used on the portions of the face and/or body on which one wants to achieve a lasting highlighted effect. The products may be provided in various viscosities, formulas, applicators and sizes. Further, the system may include a blending tool such as a brush, sponge etc.

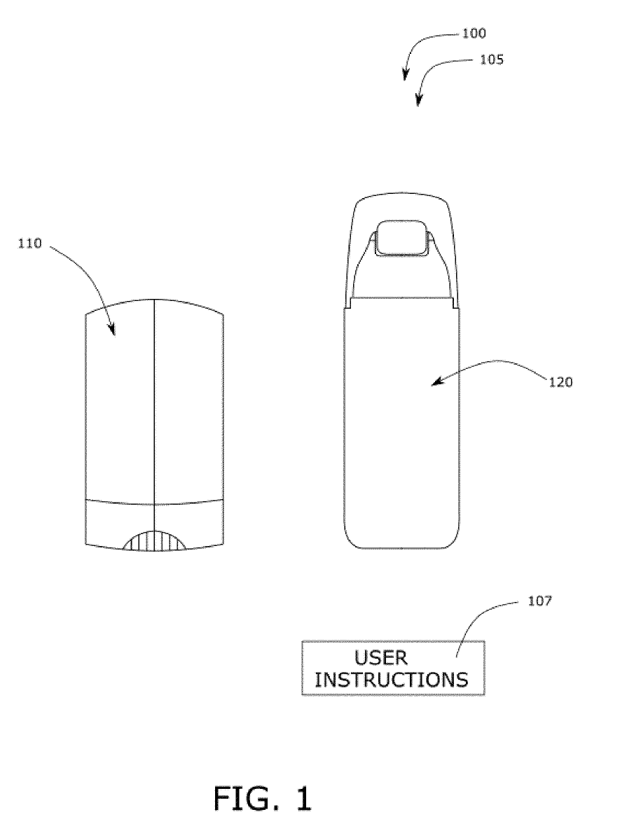

Referring now more specifically to the drawings by numerals of reference, there is shown in FIGS. 1-5B, various views of a cosmetic system 100.

FIG. 1 shows a cosmetic system 100 during an ‘in-use’ condition 150, according to an embodiment of the present disclosure. As Illustrated, the cosmetic system 100 may include a first-applicator 110 and a second-applicator 120.

According to one embodiment, the cosmetic system 100 may be arranged as a kit 105. The kit 105 may include set of user instructions 107. The instructions 107 may detail functional relationships in relation to the structure of the cosmetic system 100 (such that the cosmetic system 100 can be used, maintained, or the like, in a preferred manner).

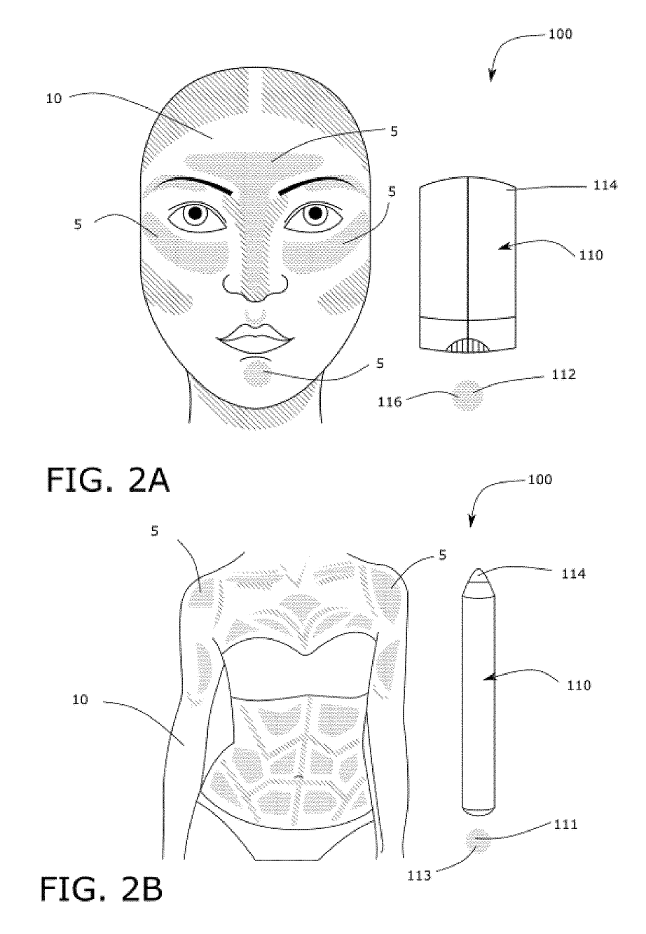

FIGS. 2A-2B show front perspective views of the cosmetic system 100 of FIG. 1, according to an embodiment of the present disclosure. The first-applicator 110 may have a first-composition 112 therein and a first applicator-end 114. The first applicator-end 114 may be configured to apply the first-composition 112 to at least one first-area 5 on at least one body-part of a user 10. Further, the first-composition 112 may include at least one sunscreen-agent 116. It should be appreciated that any present and future sunscreen-agents may be contemplated for use in the first-composition 112.

In one embodiment, the at least one sunscreen-agent 116 may comprise physical sunscreen 111. Examples of physical sunscreen 111 contemplated may include titanium dioxide, zinc oxide, etc. In another embodiment, the at least one sunscreen-agent 116 may comprise chemical sunscreen 113. Examples of chemical sunscreen 113 contemplated may include organic compounds, such as oxybenzone, octinoxate, octisalate, avobenzone, etc. The at least one sunscreen-agent 116 may comprise sun protection factor (SPF) configured to block UVB rays. In one embodiment, the SPF may be between 35-50; between 15-30; or between 55-80. However, it should be appreciated that the SPF ranges listed are not exhaustive.

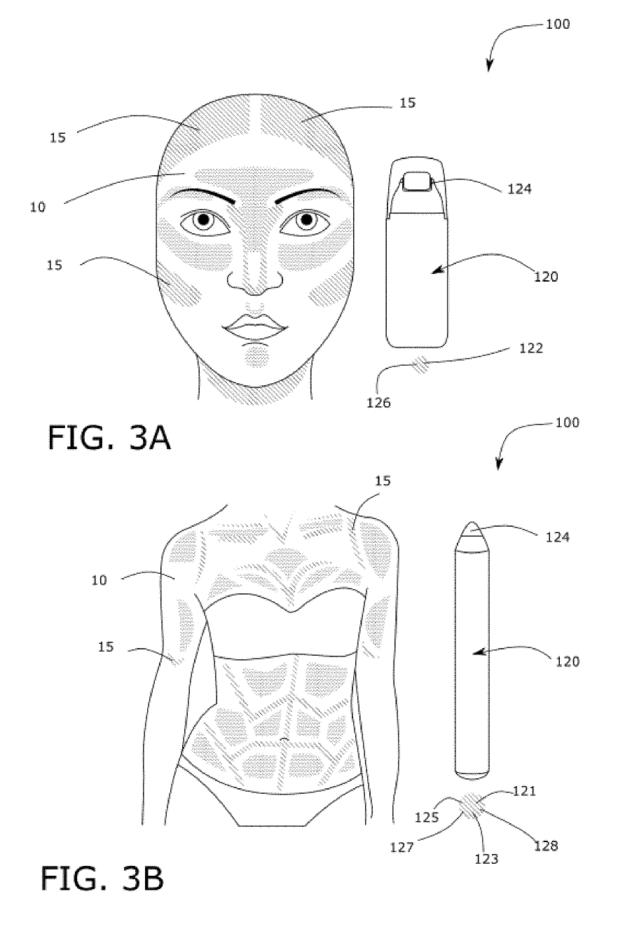

FIGS. 3A-3B show front perspective views of the cosmetic system 100 of FIG. 1, according to an embodiment of the present disclosure. The second-applicator 120 may have a second-composition 122 therein and a second applicator-end 124. The second applicator-end 124 may be configured to apply the second-composition 122 to at least one second-area 15 on the at least one body-part of the user. Further, the second-composition 122 may include at least one skin-darkening agent 126.

In one embodiment, the at least one skin-darkening agent 126 may be a temporary bronzer. In this embodiment, the temporary bronzer 121 may comprise at least one tint-ingredient 123 configured to temporarily stain the at least one second-area 15 on the at least one body-part of the user. The at least one skin-darkening agent 126 may comprise at least one self-tanning ingredient 125. In one embodiment, the at least one self-tanning ingredient 125 may comprise dihydroxyacetone 127. In another embodiment, the at least one self-tanning ingredient 125 may comprise erythrulose 128. The dihydroxyacetone 127 and the erythrulose 128 may be configured to cause a chemical reaction with amino acids located on skin surface of the user to provide a darker appearance to the at least one second-area 15 on the at least one body-part of the user.

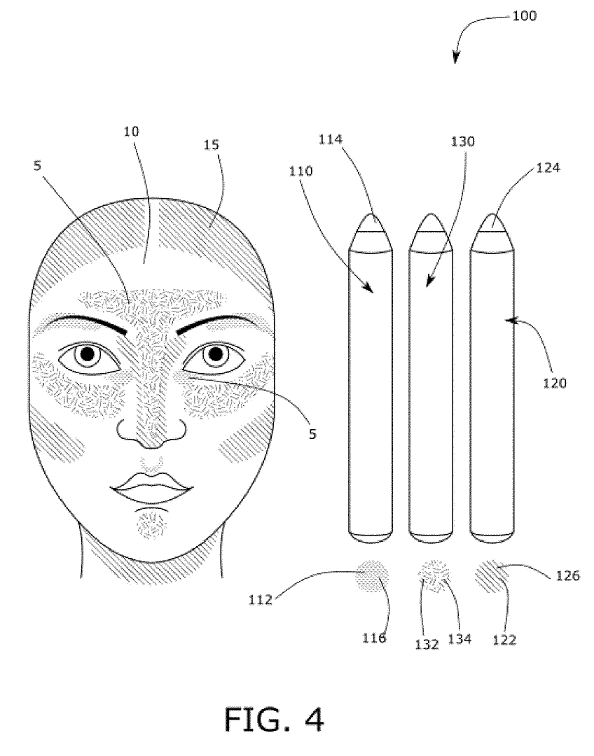

FIG. 4 shows a front perspective view of the cosmetic system 100 of FIG. 1, according to an embodiment of the present disclosure. As discussed above, the first-composition 112 may include the at least one sunscreen-agent 116. In addition to this, in one embodiment, the cosmetic system 100 may include a third applicator 130 including a third-composition 132. The third-composition 132 may comprise a second at least one sunscreen-agent 134. In one embodiment, the second at least one sunscreen-agent 134 from the third-composition 132 may include a higher sun protection factor than the at least one sunscreen-agent 116 from the first-composition 112. This may allow users to apply the third-composition 132 to higher points of the at least one first-area 5 on the at least one body-part of the user to further accentuate the highlighting to the at least one first-area 5. It should be contemplated, however, that the first-composition 112 may include the higher SPF, and the third-composition 132 may be include the lower SPF.

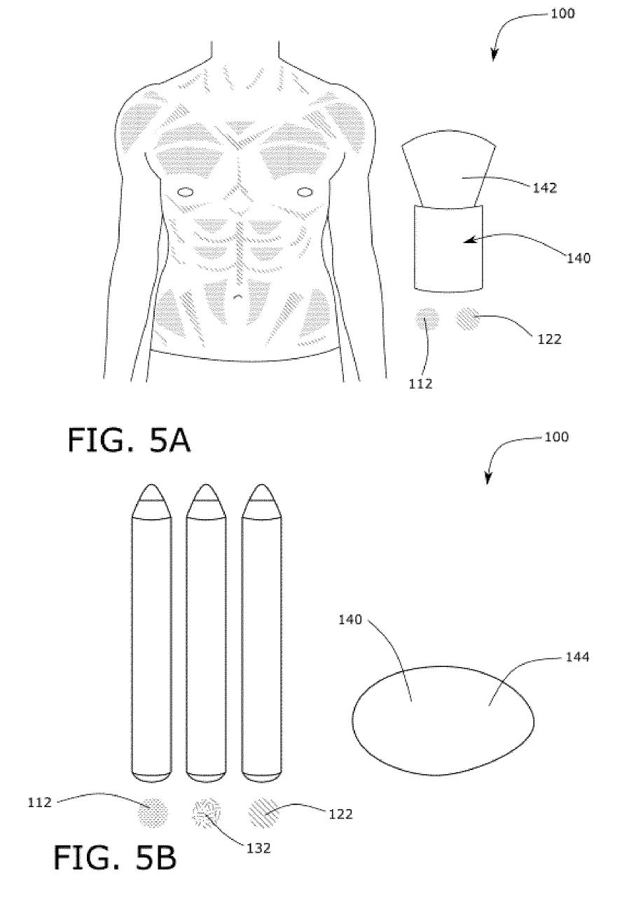

FIGS. 5A-5B show front perspective views of the cosmetic system 100 of FIG. 1, according to an embodiment of the present disclosure. As shown, the cosmetic system 100 may further include an application-means 140 configured to aid in application of at least one of the first-composition 112 and the second-composition 122 (and in some embodiments the third-composition 132). In one embodiment, the application-means 140 may be a brush 142. The brush 142 may take various forms such as kabuki brush 142, stippling brush 142, powder brush 142, blending brush 142, etc. In another embodiment, the application-means 140 may be a sponge 144. The sponge 144 may be submerged in water and rung out to avoid inadvertent absorption of the compositions, may be latex, foam, silicone, etc.

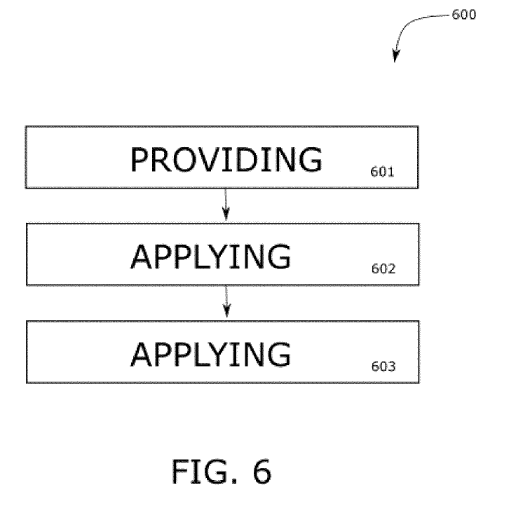

FIG. 6 is a flow diagram illustrating a method of using a cosmetic system 600, according to an embodiment of the present disclosure. As illustrated, the method of using a cosmetic system 600 may include the steps of: step one 601, providing the cosmetic system 100 as above; step two 602, applying the first-composition 112 to the at least one first-area 5 on the at least one body-part of the user; and step three 603, applying the second-composition 122 to the at least one second-area 15 on the at least one body-part of the user.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for cosmetic system 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A cosmetic system comprising:
a first-applicator having a first-composition therein and a first applicator-end, the first applicator-end configured to apply the first-composition to at least one first-area on at least one body-part of a user, and wherein the first-composition includes at least one sunscreen-agent; and
a second-applicator having a second-composition therein and a second applicator-end, the second applicator-end configured to apply the second-composition to at least one second-area on the at least one body-part of the user, and wherein the second-composition includes at least one skin-darkening agent.

2. The cosmetic system of claim 1, wherein the at least one skin-darkening agent is a temporary bronzer.

3. The cosmetic system of claim 2, wherein the temporary bronzer comprises at least one tint-ingredient configured to temporarily stain the at least one second-area on the at least one body-part of the user.

4. The cosmetic system of claim 1, wherein the at least one skin-darkening agent comprises at least one self-tanning ingredient.

5. The cosmetic system of claim 4, wherein the at least one self-tanning ingredient comprises dihydroxyacetone.

6. The cosmetic system of claim 4, wherein the at least one self-tanning ingredient comprises erythrulose.

7. The cosmetic system of claim 1, wherein the at least one sunscreen-agent comprises physical sunscreen.

8. The cosmetic system of claim 1, wherein the at least one sunscreen-agent comprises chemical sunscreen.

9. The cosmetic system of claim 1, wherein the at least one sunscreen-agent comprises a sun protection factor of between 35-50.

10. The cosmetic system of claim 1, wherein the at least one sunscreen-agent comprises a sun protection of between 15-30.

11. The cosmetic system of claim 1, wherein the at least one sunscreen-agent comprises a sun protection of between 55-80.

12. The cosmetic system of claim 1, further comprising a third-applicator including a third-composition.

13. The cosmetic system of claim 12, wherein the third-composition comprises a second said at least one sunscreen-agent.

14. The cosmetic system of claim 13, wherein the second at least one sunscreen-agent from the third-composition includes a higher sun protection factor than the at least one sunscreen-agent from the first-composition.

15. The cosmetic system of claim 1, further comprising an application-means configured to aid in application of at least one of the first-composition and the second-composition.

16. The cosmetic system of claim 15, wherein the application-means is a brush.

17. The cosmetic system of claim 15, wherein the application-means is a sponge.

18. A cosmetic system comprising:
a first-applicator including a first-composition therein and a first applicator-end, the first applicator-end configured to apply the first-composition to at least one first-area on at least one body-part of a user, and wherein the first-composition includes at least one sunscreen-agent; and
a second-applicator including a second-composition therein and a second applicator-end, the second applicator-end configured to apply the second-composition to at least one second-area on the at least one body-part of the user, and wherein the second-composition includes at least one skin-darkening agent; and
a third-applicator including a third-composition therein and a third-applicator-end, the third applicator-end configured to apply the third-composition to the at least one first-area on the at least one body-part of the user, the third-composition including a second at least one sunscreen-agent; and
an application-means configured to aid in application of the at least one of the first-composition, the second-composition and the third-composition; and
wherein the second at least one sunscreen-agent from the third-composition includes a higher sun protection factor than the at least one sunscreen-agent from the first-composition.

19. The cosmetic system of claim 18, further comprising set of instructions; and
wherein the cosmetic system is arranged as a kit.

20. A method of using a cosmetic system, the method comprising the steps of:
providing the cosmetic system, the cosmetic system including:
a first-applicator including a first-composition therein and a first applicator-end, the first applicator-end configured to apply the first-composition to at least one first-area on at least one body-part of a user, and wherein the first-composition includes at least one sunscreen-agent; and
a second-applicator including a second-composition therein and a second applicator-end, the second applicator-end configured to apply the second-composition to at least one second-area on the at least one body-part of the user, and wherein the second-composition includes at least one skin-darkening agent;
applying the first-composition to the at least one first-area on the at least one body-part of the user; and
applying the second-composition to the at least one second-area on the at least one body-part of the user.
